(19) United States
(12) Patent Application Publication
(10) Pub. No.: US 2005/0089590 A1
(43) Pub. Date: Apr. 28, 2005

(54) Method for protection of skin against sun-induced damage by oral administration of an extract of Emblica officinalis (syn. Phyllanthus emblica)

(76) Inventor: Ratan K. Chaudhuri, Lincoln Park, NJ (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/691,959

(22) Filed: Oct. 24, 2003

(51) Int. Cl. A61K 35/78

(52) U.S. Cl. 424/769

(57) ABSTRACT

A method to retard, prevent and reverse the sign of skin photo-damage, comprising administering, to a human subject in need thereof, a therapeutically effective dose of a formulation comprising an extract of Emblica officinalis as the active ingredient.

### CROSS-REFERENCE TO RELATED PATENTS

This invention is related to U.S. Pat. No. 6,124,268 to Ghosal issued Sep. 26, 2000, copending application Ser. No. 10/120,156 by Chaudhuri et al entitled “Skin Lightening” filed Apr. 11, 2002, Provisional application 60/395,612 filed Jul. 15, 2002 entitled “An Effective Method For Regulating The Appearance Of Skin” and copending application Ser. No. 10/660,742 filed Sep. 12, 2003 entitled “Enriched Aqueous Components Of Emblica officinalis.

### BACKGROUND OF THE INVENTION

1. Field of the Invention

This invention relates to sun protection, and, more particularly, to a method for sun protection of skin by oral administration of an extract of the Emblica officinalis (syn. Phyllanthus emblica) plant.

2. Description of the Prior Art

The predominant extracellular matrix component of the dermis and a variety of other human tissues is collagen, a super family of closely related, yet genetically distinct proteins. Each of the genetically distinct collagen types has an important functional role within its compartmentalized distribution in the skin.

Extrinsic aging of the skin comprises changes that represent the accumulation of the many environmental insults to the skin. The most important of these insults is obviously long-term sun exposure. The changes in collagen quantity and its structure, skin elasticity and extensibility are exaggerated in photo damaged skin. A recent study suggests that the decline in dermal collagen is greater in extrinsically photo damaged skin than in intrinsically aged skin.

In actinically damaged skin (skin damaged by the chemical action of the sun's rays), the significantly increased iron content drives the production of highly reactive oxygen species (ROS) with subsequent tissue damage and long-term consequences like cancer or premature aging of the skin. UV-inducible genes involved in this pathological degradation comprise several proteases, among them matrix-degrading metalloprotease (MMPs), which contribute to degradation of connective tissue compounds such as collagen and thus cause wrinkle formation, loss of elasticity and promote invasion and metastasis of skin cancer. The release of iron from iron-storage proteins at the cellular level due to U.V. exposure has been identified as the main source of oxidative stress.

Exposure to the sun causes premature aging of the skin. Premature aging shows up on the skin as lines, wrinkles, age spots, freckles, dryness and uneven skin tone. Photo-aging also causes collagen to break down which reduces the skin's elasticity and firmness. While these signs show that skin has been damaged, and that is a concern, it also reduces the appearance of the individual and makes them appear, in many cases, much older than they really are. This in and of itself can be very distressing, especially to women as the majority of photo-aging occurs on skin that is exposed everyday, such as the skin on the face, neck, chest, hands and arms and is therefore seen. Creams, lotions and makeup can attempt to mask the problem but cannot reverse damage caused by photo-aging. What is really needed is a product which can help protect and reverse the skin-damaging effects of the sun by nourishing and replenishing the skin from within. Until now, no such product existed, that is until the invention of this application.

Ultraviolet radiation (UVR)-induced inflammatory response is one of the prevailing mechanisms proposed to account for the majority of the UVR-dependent increases in ROS as well as UVR-dependent oxidative damage to the skin. In addition, the proinflammatory and redox-regulated transcription factor NF-ₖB has been identified as among the primary molecules targeted during the signal transduction initiated by UVR in human skin. As a result, UVR stimulates the expression of a wide variety of proinflammatory genes such as tumor necrosis factor-α (TNF-α), interleukin-1α (IL-1α), interleukin-6 (IL-6), and interleukin-8 (IL-8), which contain nuclear factor-ₖB (NF-ₖB)-binding sites in their 5′ flanking region.

Because the inflammatory response plays a substantial role in UVR-induced oxidative stress and damage in the skin through the elevation of ROS levels, antioxidants with anti-inflammatory properties are considered to counteract the damaging effects of UVR.

For example, Ghosal, S., in U.S. Pat. No. 6,124,268, described a natural antioxidant blend obtained by extraction from the Emblica officinalis plant, which could be used as a sunscreen when applied as a spray lotion, aqueous gel, or cream, to the skin of the user. This patent also describes orally administrable compositions, for example with vitamins to take advantage of the antioxidant property of the extract described therein, but with no suggestion that such composition can protect skin from sun-induced damage. Conversely, Lorenz, R., in U.S. Pat. No. 6,433,025 disclosed a method of retarding or preventing sunburns by oral administration of astaxanthin.

Accordingly, it is an object of this invention to provide a method of sun protection of skin against UVR-induced erythema by oral administration of an extract composition of the Emblica officinalis.

### SUMMARY OF THE INVENTION

What has been discovered herein is that an antioxidant composition based on an extract of the Emblica officinalis plant can provide effective sun protection of skin when taken orally by the user.

### DETAILED DESCRIPTION OF THE INVENTION

The antioxidant composition in the present invention comprises an extract of Emblica officinalis, e.g. that of U.S. Pat. No. 6,124,218 and preferably a standardized extract of low molecular weight (<1000) hydrolysable tannins, suitably over 40%, preferably 50-80%, w/w, of Emblicanin A, Emblicanin B, Pedunculagin and Punigluconin, with low levels, <1%, w/w, preferably <0.6%, of total flavonoids, which standardized extract differs from the extract described by Ghosal. The total flavonoids levels in the standardized extract does not impair the desired elegant off white-to-yellow color of the composition. In comparison, commercial competitive products, which have significantly higher contents of total flavonoids, exhibit a significantly darker color. Also, the desired concentrations of the rutin species of flavonoids (3′,4′,5′,7-tetrahydroxy-flavone-3-O-rhamnoglucoside) in the standardized extract of the invention are less than 1.0%, preferably less than 0.01%, with a value of 0.001 to 0.01% being particularly preferred. The most preferred concentrations of the components are on a percent by weight basis of the total dried extract:

Another preferred composition of the extract is described in the U.S. pending application Ser. No. 10/660,742, filed on Sep. 12, 2003. This extract composition is substantially to completely devoid of black particles when viewed visually (macroscopically), i.e. not more than 100, preferably less than 10 black particles per 500 g of composition. Additionally, this extract is substantially devoid of water-insoluble oligomeric and polymeric tannins (contains <5% w/w, preferably <1% w/w), especially such tannins having a molecular weight of over 1000 and particularly over 3000 (oligomeric/polymeric tannins). By water-insoluble it meant that a 1% by weight concentration of polymeric tannin in water does not exhibit a solubility of more than 10% by weight of the total tannin at 22° C.

The most preferred concentrations of the components are on a percent by weight basis of the total dried extract:

TABLE 1
Standardized Extract Composition

| | Most Preferred Concentrations |
|---|---|
| Component | (% by Weight) |
| Emblicanin A | 20-35 |
| Emblicanin B | 10-20 |
| Pedunculagin | 15-30 |
| Punigluconin | 3-12 |
| Total Flavonoids | <1 |

The standardized composition may exhibit average percentage deviations from these preferred values of:

TABLE 2

| | Preferred | Most Preferred |
|---|---|---|
| Component | Deviation | Deviation |
| Emblicanin A | ±10% | ±5% |
| Emblicanin B | ±10% | ±5% |
| Pedunculagin | ±10% | ±5% |
| Punigluconin | ±10% | ±5% |
| Total Flavonoids | ±10% | ±5% |

The preferred antioxidant compositions of the invention can be obtained by removal of the total flavonoids by reverse-phase column chromatography, or HPLC, using a solvent system of acetonitrile, water/phosphoric acid (20/80/1), or other solvent combinations, as they elute faster than the low molecular-weight tannins. Also, by selection of geographical location, the Phyllanthus emblica fruit extract can provide a substantially lower level of the total flavonoids (<1.0%). More particularly, it has been observed that medium-sized fruits collected from some parts of eastern India, during November-February, after water extraction and drying, yield the preferred antioxidant composition as a powder with the desired low content of total flavonoids. Accordingly, by analyzing the total flavonoids content of extracts and selecting extracts that contain the desired low content of total flavonoids, it is possible to prepare the desired standardized extract in a reproducible manner.

In the context of the present invention the term “flavonoids” include a family of compounds, which exhibit a peak at 350 nm when analyzed by UV spectral data. Examples of flavonoids include but are not limited to flavonols and flavones, a species thereof being rutin as discussed above.

The antioxidant composition of present invention is truly innovative as it uniquely provides the four features of Dermal Defense
No Pro-Oxidation Activity
Inhibition of Collagenase Activity
Iron and Copper Chelation Activity
Cascading Effect
No Pro-Oxidation Activity: To control oxidative processes, i.e. to reduce, if not prevent their harmful effects to skin, diverse antioxidants can be used to protect skin from photo-damage. When a general use of antioxidants is advocated, it is often disregarded that these compounds not only function as antioxidants, but (intrinsically) have pro-oxidant action as well, especially in the presence of transition metals like iron and copper. Release of iron from the iron-storage protein ferritin under UV-light has been ascribed to be the main source of oxidative stress. The consequent release of potentially harmful free iron within the cells will clearly exacerbate the damaging effects of photoperoxidation and is likely to be of central importance to both reversible and degenerative damage to the skin after exposure to UV light. It has been shown that the iron content of the human epidermis is three-fold greater in sun-exposed areas than in non-exposed body sites. Iron exerts its toxicity through a series of reactions with reactive oxygen species called the Fenton reaction, generating the highly toxic hydroxyl radical with subsequent damage to biomolecules. The antioxidant of the present invention is completely free of pro-oxidation activity induced by transition metals whereas well-known antioxidants like Vitamin C, Vitamin E, proanthocyanidins (from pine and grape), Superoxide Dismutase and Glutathione do exhibit pro-oxidative activity.
Inhibition of Collagenase Activity: The antioxidant of the present invention is unique in that it inhibits collagenase activity (collagenase is one of the MMP's) which digests collagen and therefore degrades collagen. Exposure to UV tends to increase the expression of the collagenase enzyme which contributes to the visible signs of aging. The antioxidant of the present invention is able to block this enzyme activity and thereby reduces the destruction of collagen.
Iron and Copper Chelation Activity: Iron is the primary growth factor for all living cells. Most of the iron in the body resides in the blodd stream. However, not all iron that enters the circulation can be carried by hemoglobin. If iron circulate through the blood stream unattached to a protein, what is called “free iron” they can wreak havoc, promoting oxidative stress. Iron-induced oxidation occuring within fatty tissues is called lipid peroxidation. For example, neurons (nerve cells) in the brain and nervous system are lined with fat called myelin and are very vulnerable to destruction by excessive levels of unbound iron.

The antioxidant of the present invention is a very efficient iron and copper chelator. Iron and copper are the principal players involved in the degredation of collagen and free radical damage which causes premature aging and photodamage to the skin. Altering the reduction potential of iron to disfavor reaction with H₂O₂ or blocking available sites on the iron to which H₂O₂ might attach may provide a solution to stop transition metal-induced oxidation. These two principles are important in the design of chelators having antioxidant functionality for skin care use. The antioxidant of the present invention has all the attributes of an ideal antioxidant but no pro-oxidant activity induced by transition metals because of its excellent chelating property for Fe³⁺ and Cu²⁺ thereby eliminating the generation of the hydroxyl radical and its detrimental effects on skin, most significantly when skin is exposed to ultraviolet light.
Cascading Effect: The antioxidant of the present invention is one of a very small group of antioxidants providing a unique “cascading effect” which potentiates free radical scavenging activity by allowing the actives to continuously recycle to remain active for a longer period of time. While most antioxidants go from an active to an inactive role, the antioxidant of the present invention utilizes a multilevel cascade of antioxidant compounds resulting in a totally unique prolongation of its antioxidant capabilities.

Nutritional supplements take many forms, varying in some instances with the intended application. They have been used in the form of liquids, pills or tablets and confectionery bars to supply diets with additional vitamins, minerals or other food groups. Protein supplements, e.g. soy proteins, are available commercially in several forms, such as powders, tablets and self-supporting solid structures. The powders are typically sprinkled on or mixed with other foods and most typically mixed with a liquid such as water or milk. Flavoring agents and other additives are typically used to make the supplement more palatable and more easily dispersed in a liquid medium. The self-supporting solid structures are available commercially, typically as confectionery bars, e.g. “candy” bars. Like their powdered counterparts, they usually contain flavoring agents and other additives intended to provide better texture and palatability. Thus, this invention contemplates all types of ingestible compositions containing Emblica and for example natural or synthetic sweeteners such as, for example, sucrose, glucose, corn syrup, fructose, maltose, dextrose, maltodextrose, calcium sorbitan, and saccharins, calcium cyclamate, aspartame, and “Splendor”.

The type of physical activity required by many types of work and athletic pursuits place greater demands on the bodies of those people involved in such endeavors, requiring consumption of high levels of certain types of nutrients. Additionally, the presence of high levels of iron and copper in sweat cause much greater oxidative stress to skin and eventual photo-aging and wrinkle formation.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLE 1

Fresh Emblica officinalis fruit (5 kg) was finely pulped and mixed with water (2-liter), containing sodium chloride (1% w/w). The mixture was left standing at room temperature for about 12 hours. Then the mixture was stored in the cold (10° C.) for 3 days. Thereafter it was filtered through a thin cloth and the filtrate was spray-dried. The antioxidant fraction in the spray-dried blend was about 0.1 g/100 g of pulp as determined by high-pressure thin layer chromatography (HPTLC). Some free gallic acid (1.8 g/100 g of pulp), and monosaccharides and starches (glucose, rhamnose, galactose, etc.) (12 g/100 g of pulp) also was present in the blend.

The following examples have not, necessarily, been conducted, but are instead illustrative of the invention.

### EXAMPLE 2

EXTRACT TABLETS AND CAPSULES

| | Composition | Quantity per |
|---|---|---|
| Ingredient | (w/w, in %) | tablet (mg) |
| 1. Extract of Invention (EX. 1) | 60.0 | 250.0 |
| 2. Avicel pH 101 | 20.0 | 84.0 |
| 3. Starch 1500 | 17.5 | 75.5 |
| 4. Stearic acid, N.F. (powder) | 2.0 | 8.5 |
| 5. Cab-O-Sil | 0.5 | 2.0 |

Note:
Extract is granulated with starch paste to make it a free-flowing powder. Blend all the ingredients, except 4, for 25 min. in a blender. Screen in 4 and blend for an additional 5 min. Compress into tablets using 7/16-in standard concave tooling. Alternately, the blended material can be filled into appropriate capsules.

### EXAMPLE 3

EXTRACT TABLETS AND CAPSULES

| | Composition | Quantity per |
|---|---|---|
| Ingredient | (w/w, in %) | tablet (mg) |
| 1. Extract of Invention (Ex. 1) | 12.26 | 27.60 |
| 2. Sodium ascorbate, USP | 36.26 | 81.60 |
| 3. Avicel pH 101 | 17.12 | 38.50 |
| 4. Sodium saccharin, (powder), N.F. | 0.56 | 1.25 |
| 5. DiPac | 29.30 | 66.00 |
| 6. Stearic acid, N.F. | 2.50 | 5.60 |
| 7. Imitation orange flavor | 1.0 | 2.25 |
| 8. FD & C Yellow #6 dye | 0.5 | 1.12 |
| 9. Cab-O-Sil | 0.5 | 1.12 |

Blend all the ingredients, except 6, for 20 min. in a blender. Screen in 4 and blend for an additional 5 min. Compress into tablets using 7/16-in standard concave tooling.

### EXAMPLE 4

EXTRACT SYRUP

| Ingredient No. | Ingredient | Quantity per 100 mL |
|---|---|---|
| 1 | Extract of Invention | 250 mg-2 gm |
| 2 | Excipients | q.s |

### EXAMPLE 5

EXTRACT BEVERAGE

| Ingredient | | Quantity per |
|---|---|---|
| No. | Ingredient | 500 mL |
| 1 | Extract of Invention | 10 mg-2 gm |
| 2 | Excipients: Carbonated Water, Food Starch- | q.s |
| | Modified, High Fructose Corn Syrup and/or | |
| | Sucrose and/or Sugar, Sodium Benzoate, | |
| | Caffeine, Glycerol Ester of Wood resin, | |
| | Flavors, Colors | |

### EXAMPLE 6

EXTRACT CEREAL

| Ingredient | | Quantity per |
|---|---|---|
| No. | Ingredient | 1 Kg |
| 1 | Extract of Invention | 500 mg-10 gm |
| 2 | Excipients: Whole Grain Oats, Oat Bran, | q.s. |
| | Sugar, Modified Corn Starch, Brown Sugar | |
| | Syrup, Salt, Calcium Carbonate, | |
| | Trisodium Phosphate, Wheat Flour, Vitamin | |
| | E (Mixed tocopherols), Zinc & Iron (Mineral | |
| | nutrients), Niacinamide (A B Vitamins), | |
| | Vitamin B6 (Pyridoxine Hcl), Vitamin B2 | |
| | (Riboflavin), Vitamin B1 (Thiamin | |
| | Mononitrate), Vitamin A (Palmitate), | |
| | Vitamin A B (Folic acid), Vitamin B12, | |
| | Vitamin D | |

### EXAMPLE 7

EXTRACT NUTRITION BAR

| Ingredient | | Quantity per |
|---|---|---|
| No. | Ingredient | 50 g |
| 1 | Extract of Invention | 50 mg-250 mg |
| 2 | Excipients: Mixed Fruit Juice Concentrates | q.s |
| | and Natural Grain Dextrins, Brown Rice | |
| | Syrup, Peanut Butter, Whey Protein | |
| | Concentrate, Peanut Flour, Agave Nectar, | |
| | Honey, Rice Flour, Calcium Caseinate, | |
| | Natural Flavors, Salt, | |
| | Whey, Flax Seeds, Soy Protein Isolate, | |
| | Lecithin, Canola Oil, Vitamin E (Mixed | |
| | tocopherols). | |

### EXAMPLE 8

### Metal Chelation Ability of Extract of Invention

Iron-catalyzed formation of hydroxyl radical from superoxide anion radical and hydrogen peroxide requires the availability of at least one iron coordination site that is either empty or occupied by a readily dissociable ligand, such as water. This coordination with water may be completely displaced by stronger ligands like azide (N₃⁻) anion. This principle was applied to determine if any coordination site is free in the Fe³⁺-antioxidant complex by UV spectrophotometric method (E. Graf et al. “Iron-catalyzed hydroxyl radical formation, stringent requirement for free iron coordination site” J. Biol. Chem. 1984 259:3620-3624.; A. E. Martell et al., in Advances in Catalysis, IX (A. Farakas, ed.), Academic Press, New York, N.Y., 319, 1957). Of all the Fe³⁺ chelates tested, only the Extract of Invention lack water in the coordination sphere (that is, the complex is fully and stably saturated and incapable of pro-oxidant activity via the formation of oxo-ferryl radical). All other antioxidants/chelators showed disparate coordination site(s) thereby allowing the formation of oxo-ferryl radical and manifesting a pro-oxidant effect, particularly at low concentrations. The Extract of Invention, being the most effective iron chelator, would prevent oxidative stress-induced damage caused by radicals and loose transition metal ions.

The results are recorded in Table 3 below:

TABLE 3
Ultraviolet Spectral Data of Fe³⁺ - Chelators*

| Chelator/ | Absorption Maxima of Complex (λₘₐₓ in nm) | |
|---|---|---|
| Antioxidant | With Fe³⁺ | N₃ Induced Shift |
| EDTA | 241, 283 | 241, 283, 410 |
| Extract of Invention | 241, 294, 353, 377 | 241, 294, 353, 377/No Shift |
| Pine Antioxidant | 241, 294, 353, 384 | 241, 294, 353, 400, 440 |
| Vitamin C | 238, 262 | 241, 266, 295 |
| Grape Antioxidant | 247, 295, 353, 396 | 247, 295, 353, 415, 430 |
| Gree Tea | 240, 272, 324, 390 | 240, 277, 325, 390 |
| Antioxidant | | |
| Trolox C | 240, 284 | 240, 273, 284, 360 |
| Gallic Acid | 247, 295, 337 | 247, 295, 353, 412 |

*The peak positions are obtained from differential spectroscopic scans of 1.0 mM Fe³⁺ and 5 mM chelator, 1 M NaN₃, 50 mM phosphate buffer, pH 7.4, vs. the same solution without sodium azide

TABLE 4
Ultraviolet Spectral Data of Cu²⁺ - Chelators*

| Chelator/ | Absorption Maxima of Complex (λₘₐₓ in nm) | |
|---|---|---|
| Antioxidant | With Cu²⁺ | N₃ Induced Shift |
| EDTA | 240, 278 | 241, 279, 354 |
| Extract of Invention | 240, 272, 313 | 240, 272, 313/No Shift |
| Pine Antioxidant | 239, 279, 302, 331 | 239, 280, 307, 430 |
| Vitamin C | 239, 263 | 239, 263, 284, 364 |
| Grape Antioxidant | 240, 277, 328 | 240, 277, 328, 359 |
| Trolox C | 241, 288 | 241, 261, 352, 440 |
| Gallic Acid | 240, 258, 321 | 240, 258, 331, 463 |

*The peak positions are obtained from differential spectroscopic scans of 1.0 mM Cu²⁺ and 5 mM chelator, 1 M NaN₃, 50 mM phosphate buffer, pH 7.4, vs the same solution without sodium azide.

### EXAMPLE 9

Contemplated Test Protocol for Evaluation and Comparison of the Extract Composition of Invention to Alter UV-Induced Erythema by Oral Administration

Specifically, this extract food supplement, after oral administration for 8 weeks, in an amount of 1-500 mg/day should increase the UV radiation necessary to produce a minimal erythemal dose (MED). Erythema would then be evaluated by expert visual assessment. Minolta Chromameter readings would be performed on baseline skin before and after 4 and 8 weeks of usage to evaluate for skin lightening potential of the extract food supplement.

1. Inclusion Criteria

a. 36 healthy male and female subjects of skin types II-III (described below) would be selected for the study.

| Skin Type | Sunburn and Tanning History |
|---|---|
| I | Always burns easily; never tans (sensitive) |
| II | Always burns easily; tans minimally (sensitive) |
| III | Burns moderately; tans gradually (normal) |
| IV | Burns minimally; always tans well (normal) |
| V | Rarely burns; tans profusely (insensitive) |
| VI | Never burns; deeply pigmented (insensitive) |

b. Ages 18-60 years old.
c. Absence of any visible skin disease which might be confused with a skin reaction from the test material.
   2. Exclusion Criteria
a. Subjects with a history of abnormal response to sunlight or those taking medication, which might produce an abnormal response to sunlight, are excluded from the study.
b. Subjects exhibiting current sunburn, suntan, uneven skin tone or visible skin disease, which might interfere with evaluation of test results, are excluded from the study.
c. Pregnant or lactating females are excluded.
d. Subjects who regularly use UVA sunbeds.
e. Subjects with a history of lupus, erythematosis, or skin cancer.
f. Subjects who are taking any vitamin supplements within 2 weeks prior to the start of the study.
   3. Light Source

A Xenon Arc Solar Simulator (150 w) would be used as the source of ultraviolet light irradiation (Solar Light Company, Philadelphia, Pa.). This instrument, described in detail in J. Invest. Dermatol. 53, 192 (1969), provides a spectral output in the ultraviolet range comparable to that of natural sunlight. WG-320 and UG-11 filters are used to provide a basic UV-A and UV-B wavelength spectrum, with wavelength ranges of 290-400 nm.

The lamp output would be measured with a UV intensity meter (Model PMA 2100 with a UVB detector, Solar Light Company, Philadelphia, Pa.) before and after the test period.

4. METHODOLOGY

The study would be performed by randomized, controlled trials with 36 volunteers randomly selected and divided into 2 groups consisting of 18 subjects each. 18 would be assigned to the orally administered group and 18 to the control group.

5. Baseline

One-week prior to the start of the study, and again on the first day of the study, the subjects would arrive at the laboratory for determination of their MED values and the mean of these two values would be taken as the baseline MED value.

Minimal Erythemal Dose (MED)

The MED is defined as the time interval or dosage of UV light irradition sufficient to produce a minimal, perceptible erythema on untreated skin. The MED of each subject would be determined by a progressive sequence of timed UV light exposures, each of which would be graduated incrementally by 25% over that of the previous exposure.

16 to 24 hours after irradiation, the sites would be evaluated for erythema according to the following scoring system:
0 Negative, no visible reaction
0.5 Minimal erythema
1 Defined erythema
2 Moderate erythema
3 Severe erythema.

In addition to the visual assessments of the MED, readings of Baseline skin would be taken using the Minolta Chroma Meter. The L*a*b* color notation system would be used to evaluate if there is a change in color over the duration of the study. Measurements would be made in triplicate and the average would be used as the data point.

Subjects in the treated groups would be instructed to administer orally 2 tablets of the extract composition of invention twice daily for 8 weeks. Subjects would be instructed to remain out of the sun and to keep a daily diary to document compliance.

Week 2

The subjects would then return to the laboratory for determination of MED values. The subjects would be irradiated as described above adjacent to the Baseline MED. The MED would be evaluated 16 to 24 hours after irradiation using the scoring system listed above.

Week 4

The subjects would then return to the laboratory for determination of MED values. The subjects would be irradiated as described above adjacent to the Baseline MED. Minolta Chroma Meter readings would be taken on Baseline skin as described above.

The MED would be evaluated 16 to 24 hours after irradiation using the scoring system listed above.

Week 8

The subjects would return to the laboratory for determination of MED values. The subjects then would be irradiated as described above adjacent to the Baseline MED. Minolta Chroma Meter readings would be taken on Baseline skin as described above.

The MED would be evaluated 16 to 24 hours after irradiation using the scoring system listed above.

At the conclusion of the study, the pre- and post-study MED's as well as the Chroma Meter readings would be compared to determine if any significant changes are observed.

It is believed that results of the protocol described above would establish scientifically the effectiveness of oral administration of the extract composition of the invention on control or protection of skin upon exposure to UV radiation.

The efficacy of protection is not comparable, however, with the use of sunscreen with a broad and high sun protection factor, but dietary supplement of the present invention may be used to increase the basal protection and thus increase the defense against UV-light mediated damage to skin. The present invention may be complemented with regular use of sunscreens applied topically, especially a sunscreen containing an extract of Emblica officinalis, especially the standardized extract. Accordingly, one aspect of the invention is to provide a regimen wherein a person will administer a topical sunscreen to the person's skin and before and/or during exposure to sun, the person will ingest a composition containing an extract of Emblica officinalis, preferably a standardized extract. Preferred regimens comprises orally administering the extract-containing composition before sun exposure, for example at least one week, or at least 2 or 3 days before sun exposure. The contemplated dosage is a sufficient to ameliorate damage to skin from exposure to sun, e.g. 1-500, preferably 2-200 mg of the standardized extract per day.

The preceding examples can be repeated with similar success by substituting the generically or specifically described reactants and/or operating conditions of this invention for those used in the preceding examples.

The entire disclosure of all applications, patents and publications, cited above and below, including but not limited to U.S. Pat. No. 6,124,268 issued Sep. 26, 2000, copending application Ser. No. 10/120,156 filed Apr. 11, 2002, copending application Ser. No. 10/660,742 filed Sep. 12, 2003, and Provisional application 60/395,612 filed Jul. 15, 2002 are hereby incorporated by reference.

From the foregoing description, one skilled in the art can easily ascertain the essential characteristics of this invention and, without departing from the spirit and scope thereof, can make various changes and modifications of the invention to adapt it to various usages and conditions.

1. A method to retard, prevent and reverse the sign of skin photo-damage, comprising administering, to a human subject in need thereof, a therapeutically effective dose of a formulation comprising an extract of Emblica officinalis as the active ingredient.

2. A product according to claim 1 in which said extract is present in a dosage form suitable for oral administration.

3. A product according to claim 2, which includes excipients suitable for such oral administration.

4. A product according to claim 2 wherein the dosage form is a tablet.

5. A product according to claim 2 wherein the said dosage form is a capsule.

6. A product according to claim 2 wherein the said dosage form is an elixir or suspension.

7. A product according to claim 2 wherein the said dosage form is a drink.

8. A product according to claim 2 wherein the said dosage form contains other ingredient capable of retarding, preventing and reversing the sign of skin photo-damage.

9. The method according to claim 1 wherein said therapeutically effective dose is in the range of about 1 to 500 mg of said extract per day.

10. The method according to claim 9 wherein said therapeutically effective dose is in the range of about 2 to 200 mg of said extract per day.

11. The method according to claim 1 wherein administration of said therapeutically effective dose is begun before, during or after sun exposure.

12. The method according to claim 11 wherein administration of said therapeutically effective dose is begun two to three days before sun exposure.

13. The method according to claim 11 wherein administration of said therapeutically effective dose is begun a week before sun exposure.

14. The method according to claim 11 wherein said extract includes Emblicanin A, Emblicanin B, Pendunculagin and Punigluconin.

15. The method according to claim 14 wherein total flavonoids are present in the extract in an amount <1% by weight of the extract.

16. The method according to claim 14 wherein said extract includes >40% by weight of said compounds.

17. The method according to claim 16 wherein rutin species of flavonoids are present in an amount of only 0.0001 to 0.01%.

18. A product according to claim 1 in which said extract is present as a nutritional supplement in the form of liquids, powders, pills or tablets, or confectionery bars.

19. A nutritional supplement for producing a baked, edible, high protein product comprising a) at least 0.1% Emblica officinalis extract, b) a mixture of high protein components, c) flour, d) leavening agent, e) sweetener, and f) water.

20. A nutritional composition according to claim 19, further including a flavor component for imparting a characteristic taste to said nutritional composition selected from the group consisting of water soluble natural or artificial extracts that include apple, banana, cherry, cinnamon, cranberry, grape, honeydew, honey, kiwi, lemon, lime, orange, peach, peppermint, pineapple, raspberry, tangerine, watermelon, wild cherry and equivalents thereof; being in the overall range of 0.10% to 2.0% by weight of said dry composition.

21. A nutritional composition according to claim 19, further including a colorant component for imparting a characteristic color to said nutritional composition selected from the group consisting of water soluble natural or artificial dyes of blue, green, orange, red, violet, and yellow; iron oxide dyes, ultramarine pigments of blue, pink, red, and violet; and equivalents thereof; being in the overall range of 0.10% to 2.0% by weight of said dry composition.

22. A method according to claim 11, further comprising topically administering a sunscreen to the skin of a human subject during sun exposure.

23. A method according to claim 12, further comprising topically administering a sunscreen to the skin of a human subject during sun exposure.

24. A method according to claim 13, further comprising topically administering a sunscreen to the skin of a human subject during sun exposure.

25. A method according to claim 1, wherein said extract is a standardized extract.

26. An ingestible nutritional composition comprising at least 0.1% Emblica officinalis extract and at least one sweetener, said composition being in the form of liquids, powders, pills or tablets, or confectionery bars.

27. An ingestible nutritional composition according to claim 26, further comprising an ingestible protein.
